(19) United States
(12) Patent Application Publication
(10) Pub. No.: US 2025/0208149 A1
(43) Pub. Date: Jun. 26, 2025

(54) WEE1 COMPOUND FOR TREATING UTERINE SEROUS CARCINOMA

(71) Applicant: Recurium IP Holdings, LLC, San Diego, CA (US)

(72) Inventors: Olivier Harismendy, San Diego, CA (US); Nathan Meade Jameson, San Diego, CA (US); Petrus Rudolf de Jong, San Diego, CA (US); Wen Liu, San Diego, CA (US); Laure Escoubet, San Diego, CA (US); Kevin Duane Bunker, Escondido, CA (US)

(21) Appl. No.: 19/045,327

(22) Filed: Feb. 4, 2025

Related U.S. Application Data
(63) Continuation of application No. PCT/US2023/071666, filed on Aug. 4, 2023.
(60) Provisional application No. 63/370,580, filed on Aug. 5, 2022.

(51) Int. Cl. G01N 33/68; A61K 31/34; A61K 31/519; A61P 35/00; G01N 33/94

(52) U.S. Cl. CPC G01N 33/6896; A61K 31/34; A61K 31/519; A61P 35/00; G01N 33/94

(57) ABSTRACT

Disclosed herein are methods of identifying or selecting subjects having a sensitivity to WEE1 inhibitors e.g., ZN-c3 and using a WEE1 inhibitor e.g., ZN-c3 to treat or inhibit a cancer including non-small cell lung cancer, breast cancer, colorectal cancer, ovarian cancer, endometrial cancer, or uterine serous carcinoma (USC).

### INCORPORATION BY REFERENCE TO PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/370,580, filed Aug. 5, 2022, which is hereby expressly incorporated herein by reference in its entirety. The present application is a continuation of PCT/US2023/071666, filed Aug. 4, 2023, which claims priority to U.S. Provisional Application Ser. No. 63/370,580, filed Aug. 5, 2022, all of which are hereby expressly incorporated herein by reference in their entireties.

### BACKGROUND

### Field

The present application relates generally to methods of identifying or selecting subjects or individuals having a sensitivity to WEE1 inhibitors e.g., ZN-c3 and using a WEE1 inhibitor e.g., ZN-c3 to treat or inhibit a cancer including non-small cell lung cancer, breast cancer, colorectal cancer, ovarian cancer, endometrial cancer, or uterine serous carcinoma (USC).

### Description

DNA is constantly damaged from the environment. Light, chemicals, stress, and cellular replication lead to single- or double-stranded breakage along DNA's backbone. Typically, organisms defend against DNA damage by repair proteins that either re-connect or re-synthesize damaged DNA. The correct functioning of these proteins is essential for life. The incorrect replacement of nucleotides into DNA can cause mutations (and other genetic alterations including but not limited to insertions, deletions, and frameshifts), genetic disease, and loss of protein function. The altogether loss of DNA repair can cause cell death, tumor progression, and cancer.

Cell cycle checkpoints are important for proper DNA repair, ensuring that cells do not progress with cellular replication until their genomic integrity is restored. WEE1 is a nuclear kinase involved in the G2-M cell-cycle checkpoint arrest for DNA repair before mitotic entry. Normal cells repair damaged DNA during G1 arrest. Cancer cells often have a deficient G1-S checkpoint and depend on a functional G2-M checkpoint for DNA repair. WEE1 is overexpressed in various cancer types, and a number of inhibitors and/or degraders of WEE1 are known to those skilled in the art. See, e.g., WO 2019/173082 and WO 2020/069105.

Uterine serous carcinoma (USC) is a highly aggressive Type II endometrial cancer. See, e.g., Ferriss J S et al, Uterine serous carcinoma: key advances and novel treatment approaches. International Journal of Gynecologic Cancer 2021; 31:1165-1174. A phase II study of adavosertib in recurrent uterine serous carcinoma has been reported. See Liu J F et al. Phase II study of the WEE1 inhibitor adavosertib in recurrent uterine serous carcinoma. J Clin Oncol 2021; 39. However, existing therapeutic approaches to treat USC and other cancers sensitive to WEE 1 inhibitors remain limited, and thus there is an urgent need for additional therapies and approaches to identify or select individuals that would benefit from such therapies.

### SUMMARY

Various embodiments described herein concern methods of providing a therapy e.g., administration of the drug ZN-c3, to a subject or individual having a cancer e.g., a cancer sensitive to a WEE 1 inhibitor, such as non-small cell lung cancer, breast cancer, colorectal cancer, ovarian cancer, endometrial cancer, or uterine serous carcinoma (USC), and methods of identifying or selecting an individual or subject that would benefit from receiving such a therapy. The methods described herein can include administration of a therapeutically effective amount of a WEE 1 inhibitor, such as the drug ZN-c3, or a pharmaceutically acceptable salt thereof, to a subject or individual in need thereof and may additionally include screening said subject or individual for a modulation in the protein phosphatase 2 scaffold subunit Alpha (PPP2R1A) protein or a gene encoding said PPP2R1A protein, for instance a polymorphism, which confers a sensitivity to a WEE 1 inhibitor, e.g., the drug ZN-c3, such as a polymorphism selected from P179R, S256F or R183W or a nucleic acid encoding a polymorphism selected from P179R, S256F or R183W, in a biological sample obtained from said subject or individual. Methods of identifying or selecting a subject or individual that has a sensitivity to a WEE 1 inhibitor, e.g., the drug ZN-c3, are contemplated whereby a biological sample from said subject or individual is analyzed to determine the presence or absence of a modulation in the protein phosphatase 2 scaffold subunit Alpha (PPP2R1A) protein or a gene encoding said PPP2R1A protein, such as a polymorphism, which confers a sensitivity to a WEE 1 inhibitor e.g., the drug ZN-c3, such as a polymorphism selected from P179R, S256F or R183W or a nucleic acid encoding a polymorphism selected from P179R, S256F or R183W are also contemplated. These and other embodiments are described in greater detail below.

1. A method of identifying or selecting a subject that has a sensitivity to the drug ZN-c3, comprising: identifying a modulation in the protein phosphatase 2 scaffold subunit Alpha (PPP2R1A) protein or a gene encoding said PPP2R1A protein, such as one or more polymorphisms, which confers a sensitivity to the drug ZN-c3, preferably a polymorphism selected from P179R, S256F or R183W or a nucleic acid encoding a polymorphism selected from P179R, S256F or R183W, in a biological sample obtained from said subject or individual; and selecting or identifying said subject as one having a sensitivity to the drug ZN-c3, when said modulation, such as said any one or more polymorphisms in the PPP2R1A gene or protein, is identified.

2. The method of alternative 1, further comprising administering ZN-c3 to said subject when said modulation or polymorphism is identified in said biological sample.

3. The method of any one of alternatives 1 or 2, wherein said subject has a cancer.

4. The method of alternative 3, wherein the cancer comprises non-small cell lung cancer, breast cancer, colorectal cancer, ovarian cancer, uterine serous carcinoma (USC), or endometrial cancer.

5. The method of any one of alternatives 2-4, wherein regression or inhibition of said cancer is greater than 7%, such as 7%. 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

6. The method of any one or alternatives 2-5 further comprising administration of an agent or combination of agents to said subject, which inhibit the PPP2R1A gene or protein.

7. The method of alternative 6, wherein said agent or combination of agents comprise the PP2A catalytic inhibitor LB-100.

8. The method of any one of alternatives 1 through 7, wherein the modulation of PPP2R1A comprises one or more gain of function mutations.

9. The method of any one of alternatives 1 through 7, wherein the modulation of PPP2R1A comprises one or more loss of function mutations.

10. The method of any one of alternatives 1 through 7, wherein the modulation of PPP2R1A comprises one or more mutations that result in overexpression of PPP2R1A.

11. The method of any one of alternatives 1 through 7, wherein the modulation of PPP2R1A comprises one or more mutations that result in under expression of PPP2R1A.

12. A method of inhibiting, ameliorating, or treating a cancer or sequela thereof in a subject, the method comprising: identifying a modulation in the protein phosphatase 2 scaffold subunit Alpha (PPP2R1A) protein or a gene encoding said PPP2R1A protein, wherein said polymorphism confers a sensitivity to the drug ZN-c3, such as a polymorphism selected from P179R, S256F or R183W or a nucleic acid encoding a polymorphism selected from P179R, S256F or R183W, in a biological sample obtained from said subject; and administering ZN-c3 to said subject when said polymorphism is identified in said biological sample.

13. The method of alternative 12, wherein the cancer comprises non-small cell lung cancer, breast cancer, colorectal cancer, ovarian cancer, uterine serous carcinoma (USC), or endometrial cancer.

14. The method of alternative 12 or 13, wherein regression or inhibition of said cancer is greater than 7%, such as 7%. 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

15. The method of any one of alternatives 12-14, wherein an agent or combination of agents, which inhibit the PPP2R1A gene or protein are additionally provided to said subject.

16. The method of alternative 15, wherein said agent or combination of agents comprise the PP2A catalytic inhibitor LB-100.

17. The method of any one of alternatives 12 through 16, wherein the modulation of PPP2R1A comprises one or more gain of function mutations.

18. The method of any one of alternatives 12 through 16, wherein the modulation of PPP2R1A comprises one or more loss of function mutations.

19. The method of any one of alternatives 12 through 16, wherein the modulation of PPP2R1A comprises one or more mutations that result in overexpression of PPP2R1A.

20. The method of any one of alternatives 12 through 16, wherein the modulation of PPP2R1A comprises one or more mutations that result in under expression of PPP2R1A.

21. The compound ZN-c3 for use in inhibiting, ameliorating, or treating a cancer or sequela thereof in a subject identified as having a modulation in the protein phosphatase 2 scaffold subunit Alpha (PPP2R1A) protein or a gene encoding said PPP2R1A protein, such as a polymorphism, which confers a sensitivity to the drug ZN-c3, e.g., a polymorphism selected from P179R, S256F or R183W or a nucleic acid encoding a polymorphism selected from P179R, S256F or R183W, in a biological sample obtained from said subject.

22. The compound ZN-c3 for use according to alternative 21, wherein the cancer comprises non-small cell lung cancer, breast cancer, colorectal cancer, ovarian cancer, uterine serous carcinoma (USC), or endometrial cancer.

23. The compound ZN-c3 for use according to alternative 21, wherein regression or inhibition of said cancer is greater than 7%, such as 7%. 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

24. The compound ZN-c3 for use according to any one of alternatives 21-23, wherein an agent or combination of agents, which inhibit the PPP2R1A gene or protein are additionally provided to said subject.

25. The compound ZN-c3 for use according to alternative 24, wherein said agent or combination of agents comprise the PP2A catalytic inhibitor LB-100.

26. The compound ZN-c3 for use according to any one of alternatives 21 through 25, wherein the modulation of PPP2R1A comprises one or more gain of function mutations.

27. The compound ZN-c3 for use according to any one of alternatives 21 through 25, wherein the modulation of PPP2R1A comprises one or more loss of function mutations.

28. The compound ZN-c3 for use according to any one of alternatives 21 through 25, wherein the modulation of PPP2R1A comprises one or more mutations that result in overexpression of PPP2R1A.

29. The compound ZN-c3 for use according to any one of alternatives 21 through 25, wherein the modulation of PPP2R1A comprises one or more mutations that result in under expression of PPP2R1A.

30. A method of inhibiting, ameliorating, or treating a cancer or sequela thereof in a subject, the method comprising: administering an agent or combination of agents, which inhibit the PPP2R1A gene or protein, to said subject; and administering ZN-c3 to said subject.

31. The method of alternative 30, wherein said agent or combination of agents, which inhibit the PPP2R1A gene or protein, are provided to said subject separately.

32. The method of alternative 30 or 31, wherein said agent or combination of agents, which inhibit the PPP2R1A gene or protein, are provided to said subject before administration of ZN-C3.

33. The method of alternative 30, wherein said agent or combination of agents, which inhibit the PPP2R1A gene or protein, are provided to said subject within 1, 2, 3, 4, 5, 6, 7, 8, 9, 10, 11, 12, 13, 14, 15, 16, 17, 18, 19, or 20 minutes apart.

34. The method of any one of alternatives 30-33, wherein the cancer comprises non-small cell lung cancer, breast cancer, colorectal cancer, ovarian cancer, uterine serous carcinoma (USC), or endometrial cancer.

35. The method of any one of alternatives 30-34, wherein regression or inhibition of said cancer is greater than 7%, such as 7%. 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

36. The method of any one of alternatives 30-35, wherein said agent or combination of agents comprise the PP2A catalytic inhibitor LB-100.

37. A product combination comprising the compound ZN-c3 and an agent or combination of agents, which inhibit the PPP2R1A gene or protein for use in inhibiting, ameliorating, or treating a cancer or sequela thereof in a subject.

38. The product combination for use according to alternative 37, wherein the cancer comprises non-small cell lung cancer, breast cancer, colorectal cancer, ovarian cancer, uterine serous carcinoma (USC), or endometrial cancer.

39. The product combination for use according to any one of alternatives 37 or 38, wherein regression or inhibition of said cancer is greater than 7%, such as 7%. 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

40. The product combination for use according to any one of alternatives 37-39, wherein said agent or combination of agents comprise the PP2A catalytic inhibitor LB-100.

41. The method of any one of alternatives 1-20 or the compound ZN-c3 for use according to any one of alternatives 21-29, wherein the presence of said polymorphism is determined using Next Generation Sequencing (NGS), sequencing, Polymerase Chain Reaction (PCR), Loop-mediated isothermal amplification, Recombinase polymerase amplification, or antibody detection.

### BRIEF DESCRIPTION OF THE DRAWINGS

FIG. 1 depicts the percent cell density of A427 (a first non-small cell lung cancer cell line) cells after treatment with a 10-point dose response of ZN-c3 for 72 hours, comparing cells transfected with siRNA to PPP2R1A against those transfected with a scrambled control siRNA. Percent cell density is calculated using cell titer glow analysis. PPP2R1A siRNA transfected cells have approximately 3.5-fold increase in sensitivity to ZN-c3 (TABLE 1).

FIG. 2 depicts the mRNA expression of PPP2R1A in A427 cells treated with either scrambled control siRNA or siRNA against PPP2R1A. mRNA is expressed as a percentage of scrambled control.

FIG. 3 depicts the percent cell density of H1975 (a second non-small cell lung cancer cell line) cells after treatment with a 10-point dose response of ZN-c3 for 72 hours, comparing cells transfected with siRNA to PPP2R1A against those transfected with a scrambled control siRNA. Percent cell density is calculated using cell titer glow analysis. PPP2R1A siRNA transfected cells have approximately 3.7-fold increase in sensitivity to ZN-c3 (TABLE 2).

FIG. 4 depicts the mRNA expression of PPP2R1A in H1975 cells treated with either scrambled control siRNA or siRNA against PPP2R1A. mRNA is expressed as a percentage of scrambled control.

FIG. 5 depicts the percent cell density of Hs578T (a breast cancer cell line) cells after treatment with a 10-point dose response of ZN-c3 for 72 hours, comparing cells transfected with siRNA to PPP2R1A against those transfected with a scrambled control siRNA. Percent cell density is calculated using cell titer glow analysis. PPP2R1A siRNA transfected cells have approximately 2.4-fold increase in sensitivity to ZN-c3 (TABLE 3).

FIG. 6 depicts the mRNA expression of PPP2R1A in Hs578T cells treated with either scrambled control siRNA or siRNA against PPP2R1A. mRNA is expressed as a percentage of scrambled control.

FIG. 7 depicts the percent cell density of SW620 (a colorectal cancer cell line) cells after treatment with a 10-point dose response of ZN-c3 for 72 hours, comparing cells transfected with siRNA to PPP2R1A against those transfected with a scrambled control siRNA. Percent cell density is calculated using cell titer glow analysis. PPP2R1A siRNA transfected cells have approximately 1.9-fold increase in sensitivity to ZN-c3 (TABLE 4).

FIG. 8 depicts the mRNA expression of PPP2R1A in SW620 cells treated with either scrambled control siRNA or siRNA against PPP2R1A. mRNA is expressed as a percentage of scrambled control.

FIG. 9 depicts the percent cell density of SKOV3 (an ovarian cancer cell line) cells after treatment with a 10-point dose response of ZN-c3 for 72 hours, comparing cells transfected with siRNA to PPP2R1A against those transfected with a scrambled control siRNA. Percent cell density is calculated using cell titer glow analysis. PPP2R1A siRNA transfected cells have approximately 2.7-fold increase in sensitivity to ZN-c3 (TABLE 5).

FIG. 10 depicts the mRNA expression of PPP2R1A in SKOV3 cells treated with either scrambled control siRNA or siRNA against PPP2R1A. mRNA is expressed as a percentage of scrambled control.

FIG. 11 depicts the percent cell density of HEC1B and HEC-59 (endometrial cancer cell lines) cells after treatment with a 10-point dose response of ZN-c3 for 72 hours, comparing cells transfected with siRNA to PPP2R1A against those transfected with a scrambled control siRNA. HEC1B has missense mutations (W257L; L429F) in PPP2R1A rendering the siRNA to PPP2R1A less effective at sensitizing to ZN-c3. HEC-59 is PPP2R1A wild-type. Percent cell density is calculated using cell titer glow analysis. HEC1B PPP2R1A siRNA transfected cells have approximately 1.4-fold increase in sensitivity to ZN-c3 while HEC-59 PPP2R1A siRNA transfected cells have approximately 2.5 fold increase in sensitivity to ZN-c3 (TABLE 6).

FIG. 12 depicts a Western blot analysis of endometrial cancer cell lines HEC1B and HEC-59 with and without siRNA mediated knockdown of PPP2R1A. Vinculin is used as a loading control.

FIG. 13 depicts the percent cell density of SKOV3 ovarian cancer cells after treatment with a 10-point dose response of ZN-c3 for 72 hours, comparing cells transduced with a non-targeting guide RNA to against those with genetic knockout of PPP2R1A after transduction with a CRISPR guide RNA targeting PPP2R1A. Percent cell density is calculated using cell titer glow analysis. PPP2R1A CRISPR knockout cells have approximately 1.7-fold increase in sensitivity to ZN-c3 (TABLE 7).

FIG. 14 depicts a Western blot analysis of the ovarian cancer cell line SKOV3 with and without CRISPR-mediated genetic knockout of PPP2R1A. Beta-actin is used as a loading control.

FIG. 15 depicts the percent cell density of H1975 lung cancer cells after treatment with a 10-point dose response of ZN-c3 for 72 hours, comparing cells transduced with a non-targeting guide RNA to against those with genetic knockout of PPP2R1A after transduction with a CRISPR guide RNA targeting PPP2R1A. Percent cell density is calculated using cell titer glow analysis. PPP2R1A CRISPR knockout cells have approximately 1.9 fold increase in sensitivity to ZN-c3 (TABLE 8).

FIG. 16 depicts a Western blot analysis of H1975 lung cancer cells with and without CRISPR-mediated genetic knockout of PPP2R1A. Beta-actin is used as a loading control.

FIG. 17 depicts the percent cell density of OV17R ovarian cancer cells after treatment with a 10-point dose response of ZN-c3 for 72 hours. OV17R has the S256F hotspot missense mutation in PPP2R1A. This cell line is highly sensitive to ZN-c3 treatment likely in part to the PPP2R1A mutation (TABLE 9). Percent cell density is calculated using cell titer glow analysis.

FIG. 18 depicts the percent inhibition of cell growth in HEC-59 endometrial cancer cells after treatment with ZN-c3 alone, the PP2A catalytic inhibitor LB-100 alone, or the combination of ZN-c3+LB-100. HEC-59 cell line is PPP2R1A wild-type. Single agent treatment with either compound is not as effective as combination therapy. Percent inhibition is calculated using cell titer glow analysis. Doses of each compound are shown in TABLE 10.

FIG. 19 depicts the percent inhibition of cell growth in OV17R ovarian cancer cells after treatment with ZN-c3 alone, the PP2A catalytic inhibitor LB-100 alone, or the combination of ZN-c3+LB-100. OV17R has the PPP2R1A missense mutation S256F. Percent inhibition is calculated using cell titer glow analysis. Doses of each compound are shown in TABLE 11.

FIG. 20 depicts the percent inhibition of cell growth in HEC-59 endometrial cancer cells after treatment with ZN-c3 alone, the PP2A catalytic inhibitor LB-100 alone, or the combination of ZN-c3+LB-100 after siRNA mediated knockdown of PPP2R1A. Knockdown of PPP2R1A results in increased ZN-c3 sensitivity both as single agent and as combination therapy. Percent inhibition is calculated using cell titer glow analysis. Doses of each compound are shown in TABLE 12.

FIG. 21 depicts the percent inhibition of cell growth in HEC1B endometrial cancer cells after treatment with ZN-c3 alone, the PP2A catalytic inhibitor LB-100 alone, or the combination of ZN-c3+LB-100 after siRNA mediated knockdown of PPP2R1A. Knockdown of PPP2R1A results in a minor increase in sensitivity as seen previously. Percent inhibition is calculated using cell titer glow analysis. Doses of each compound are shown in TABLE 13.

### DETAILED DESCRIPTION

WEE1 is a tyrosine kinase that is a critical component of the ATR-mediated G2 cell cycle checkpoint control that prevents entry into mitosis in response to cellular DNA damage. ATR phosphorylates and activates CHK1, which in turn activates WEE1, leading to the selective phosphorylation of cyclin-dependent kinase 1 (CDK1) at Tyr15, thereby stabilizing the CDK1-cyclin B complex and halting cell-cycle progression. This process confers a survival advantage by allowing tumor cells time to repair damaged DNA prior to entering mitosis. Inhibition of WEE1 abrogates the G2 checkpoint, promoting cancer cells with DNA damage to enter into unscheduled mitosis and undergo cell death via mitotic catastrophe. WEE1 inhibition and/or degradation has the potential to sensitize tumors to DNA-damaging agents, such as cisplatin, and to induce tumor cell death.

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The terms “WEE1 inhibition”, “WEE1 inhibitor” and similar terms as used herein refer to inhibiting the activity or function of a WEE1 tyrosine kinase, e.g., by degrading WEE1 tyrosine kinase and/or by reducing the activity of WEE1 tyrosine kinase with regard to mediating phosphorylation of CDK1. A WEE1 inhibitor that functions by degrading WEE1 tyrosine kinase may be referred to herein as a WEE1 degrader.

As used herein, a “subject” refers to an animal that is the object of treatment, observation or experiment. The subject animal may be a mammal such as, without limitation, mice, rats, rabbits, guinea pigs, dogs, cats, sheep, goats, cows, horses, primates, such as monkeys, chimpanzees, and apes, and, in particular, humans. In some embodiments, the subject can be human. In some embodiments, the subject can be a child and/or an infant. In other embodiments, the subject can be an adult.

As used herein, the terms “treat,” “treating,” “treatment,” “therapeutic,” and “therapy” do not necessarily mean total cure or abolition of the disease or condition. Any alleviation of any undesired signs or symptoms of the disease or condition, to any extent can be considered treatment and/or therapy. Furthermore, treatment may include acts that may worsen the subject's overall feeling of well-being or appearance.

The terms “therapeutically effective amount” and “effective amount” are used to indicate an amount of an active compound (e.g., ZN-c3 or pharmaceutically acceptable salt thereof), that elicits the biological or medicinal response indicated. For example, a therapeutically effective amount of such a ZN-c3 compound, salt or composition can be the amount needed to prevent, alleviate or ameliorate symptoms of the disease or condition, or prolong the survival of the subject being treated. This response may occur in a tissue, system, animal or human and includes alleviation of the signs or symptoms of the disease or condition being treated. Determination of an effective amount is well within the capability of those skilled in the art, in view of the disclosure provided herein. The therapeutically effective amount of the ZN-c3 compound, salt or composition required as a dose will depend on the route of administration, the type of animal, including human, being treated and the physical characteristics of the specific animal under consideration. The dose can be tailored to achieve a desired effect, but will depend on such factors as weight, diet, concurrent medication and other factors which those skilled in the medical arts will recognize.

For example, an effective amount of a ZN-c3 compound, salt or composition, may be the amount that results in: (a) the reduction, alleviation or disappearance of one or more symptoms caused by USC, (b) the reduction of tumor size, (c) the elimination of the tumor, and/or (d) long-term disease stabilization (growth arrest) of the tumor. As an example, an effective amount of a ZN-c3 compound, salt or composition may be the amount which results in the reduction in WEE1 activity and/or phosphorylation (such as phosphorylation of CDC2). The reduction in WEE1 activity is known to those skilled in the art and can be determined by the analysis of WEE1 intrinsic kinase activity and downstream substrate phosphorylation.

As used herein, the term “dosing regimen” refers to the manner in which the ZN-c3 compound is administered to the subject, including route of administration, amount of dose and dosing interval. A dosing regimen may comprise a “periodic” dosing phase, during which a particular dosage amount (e.g., 300 mg) is administered at regular intervals (e.g., once per day) for a particular period of time (e.g., three days). A dosing regimen may further comprise an “intermittent” dosing phase, during which one or more dosing parameters such as dosage amount and/or dosage interval are varied or changed. For example, an intermittent dosing phase may comprise a “rest” phase during which the ZN-c3 compound is not administered or is administered at a reduced dosage amount and/or less frequently.

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

In some embodiments, a method of selecting or identifying a subject that has a sensitivity to the drug ZN-c3 is provided. In some embodiments, the methods comprise: identifying or detecting a modulation in the protein phosphatase 2 scaffold subunit alpha (PPP2R1A) protein or a gene encoding said PPP2R1A protein e.g., such a modulation can be an overexpression or under expression of the PPP2R1A protein or a transcript encoding the PPP2R1A protein or can be a polymorphism in the PPP2R1A protein or a gene or transcript encoding the PPP2R1A protein. In some embodiments, a polymorphism, which confers a sensitivity to the drug ZN-c3 is identified or detected in said methods. In some such embodiments, the polymorphism is selected from P179R, S256F or R183W or a nucleic acid encoding a polymorphism selected from P179R, S256F or R183W. The aforementioned modulation the PPP2R1A protein or gene or transcript encoding the PPP2R1A protein is determined by analyzing a biological sample obtained from said subject that has a cancer such as non-small cell lung cancer, breast cancer, colorectal cancer, ovarian cancer, endometrial cancer, or uterine serous carcinoma (USC). In some embodiments, the method comprises selecting said subject as one having a sensitivity to the drug ZN-c3, when any one or more of the aforementioned polymorphisms in the PPP2R1A gene or protein are identified. In some embodiments, once a subject is identified or selected as one having a sensitivity to ZN-c3, said identified or selected subject is provided ZN-c3 e.g., is placed on a dosage regimen of ZN-c3.

In some embodiments, a method of inhibiting, ameliorating, or treating a cancer or sequela thereof in a subject is provided e.g., non-small cell lung cancer, breast cancer, colorectal cancer, ovarian cancer, endometrial cancer, or uterine serous carcinoma (USC). In some embodiments, the method comprises identifying or detecting a modulation in the protein phosphatase 2 scaffold subunit Alpha (PPP2R1A) protein or a gene encoding said PPP2R1A protein e.g., such a modulation can be an overexpression or under expression of the PPP2R1A protein or a transcript encoding the PPP2R1A protein or can be a polymorphism in the PPP2R1A protein or a gene or transcript encoding the PPP2R1A protein. In some embodiments, a polymorphism, which confers a sensitivity to the drug ZN-c3 is identified or detected in said methods. In some embodiments, the polymorphism is selected from P179R, S256F or R183W or a nucleic acid encoding a polymorphism selected from P179R, S256F or R183W, in a biological sample obtained from said subject. In some embodiments, ZN-c3 is administered to said subject when said polymorphism is identified in said biological sample.

In some embodiments, the compound ZN-c3 is provided for use in inhibiting, ameliorating, or treating a cancer or sequela thereof in a subject identified as having a modulation in the protein phosphatase 2 scaffold subunit Alpha (PPP2R1A) protein or a gene encoding said PPP2R1A protein, wherein said polymorphism confers a sensitivity to the drug ZN-c3, such as a polymorphism selected from any one or more of P179R, S256F or R183W or a nucleic acid encoding a polymorphism selected from any one or more of P179R, S256F or R183W, in a biological sample obtained from said subject. In some such uses, the cancer is e.g., non-small cell lung cancer, breast cancer, colorectal cancer, ovarian cancer, endometrial cancer, or uterine serous carcinoma (USC).

In some embodiments, a method of inhibiting, ameliorating, or treating a cancer or sequela thereof in a subject is provided e.g., non-small cell lung cancer, breast cancer, colorectal cancer, ovarian cancer, endometrial cancer, or uterine serous carcinoma (USC). In some embodiments, the method comprises administering an agent or combination of agents, which inhibit the PPP2R1A gene or protein, to said subject and administering ZN-c3 to said subject.

In some embodiments, a product combination is provided comprising the compound ZN-c3 and an agent or combination of agents, which inhibit the PPP2R1A gene or protein for use in inhibiting, ameliorating, or treating a cancer or sequela thereof in a subject e.g., non-small cell lung cancer, breast cancer, colorectal cancer, ovarian cancer, endometrial cancer, or uterine serous carcinoma (USC).

In some embodiments, the methods set forth above include administering ZN-c3 to said subject when a modulation in PPP2R1A protein or a gene or transcript encoding PPP2R1A protein such as polymorphism, preferably a polymorphism selected from any one or more of P179R, S256F or R183W or a polymorphism in a gene encoding any one or more of a polymorphism selected from P179R, S256F or R183W is identified in said biological sample.

In some embodiments, said subject has a cancer.

In some embodiments, the cancer comprises non-small cell lung cancer, breast cancer, colorectal cancer, ovarian cancer, or endometrial cancer.

In some embodiments, regression or inhibition of said cancer is greater than 7%, such as 7%. 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

In some embodiments, the methods further comprise administration of an agent or combination of agents to said subject, which inhibit the PPP2R1A gene or protein.

In some embodiments, said agent or combination of agents provided to said subject comprise the PP2A catalytic inhibitor LB-100.

In some embodiments, said agent or combination of agents, which inhibit the PPP2R1A gene or protein, are provided to said subject separately.

In some embodiments, said agent or combination of agents, which inhibit the PPP2R1A gene or protein, are provided to said subject before administration of ZN-C3.

In some embodiments, said agent or combination of agents, which inhibit the PPP2R1A gene or protein, are provided to said subject within 1, 2, 3, 4, 5, 6, 7, 8, 9, 10, 11, 12, 13, 14, 15, 16, 17, 18, 19, or 20 minutes apart.

In some embodiments, the presence of said any one or more polymorphism e.g., a polymorphism in a gene encoding P179R, S256F or R183W is determined using Next Generation Sequencing (NGS), sequencing, Polymerase Chain Reaction (PCR), Loop-mediated isothermal amplification, Recombinase polymerase amplification, or antibody detection.

In some embodiments, the modulation of PPP2R1A comprises one or more gain of function mutations.

In some embodiments, the modulation of PPP2R1A comprises one or more loss of function mutations.

In some embodiments, the modulation of PPP2R1A comprises one or more mutations that result in overexpression of PPP2R1A.

In some embodiments, the modulation of PPP2R1A comprises one or more mutations that result in under expression of PPP2R1A.

WEE1 Compound

ZN-c3 is a selective, orally bioavailable small molecule inhibitor of WEE1, a crucial component of the G2/M cell cycle checkpoint, which prevents cells from entering mitosis to allow repair of DNA damage. ZN-c3 has demonstrated significant in vitro antitumor activity in multiple cell lines and xenograft models.

| ZN-c3 Chemical Structure | ZN-c3 Chemical Name |
|---|---|
| 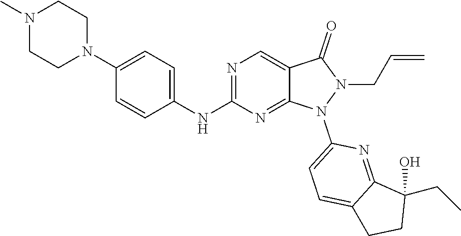 [SMILES: C=CCn1c(=O)c2cnc(Nc3ccc(N4CCN(C)CC4)cc3)nc2n1-c1ccc2c(n1)[C@@](O)(CC)CC2] | (R)-2-allyl-1-(7-ethyl-7-hydroxy-6,7- dihydro-5H-cyclopenta[b]pyridin-2-yl)-6- ((4-(4-methylpiperazin-1-yl)phenyl)amino)- 1,2-dihydro-3H-pyrazolo[3,4-d]pyrimidin- 3-one |

ZN-c3 can be prepared as described in WO 2019/173082 (see, e.g., Example 9B therein), which is hereby expressly incorporated herein by reference and particularly for the purposes of describing methods for making ZN-c3, as well as for making salts and pharmaceutical compositions thereof. If there is any discrepancy between the chemical name and structure of ZN-c3, the structure should be given more weight.

### Pharmaceutical Compositions

Some embodiments described herein relate to a pharmaceutical composition, that can include an effective amount of the ZN-c3 compound, or a pharmaceutically acceptable salt thereof, and a pharmaceutically acceptable carrier, diluent, excipient or combination thereof.

The term “pharmaceutical composition” refers to a mixture of ZN-c3 and/or pharmaceutically acceptable salts thereof, with other chemical components, such as diluents or carriers. The pharmaceutical composition facilitates administration of the compound to an organism. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, and salicylic acid. Pharmaceutical compositions will generally be tailored to the specific intended route of administration.

The term “physiologically acceptable” defines a carrier, diluent or excipient that does not abrogate the biological activity and properties of the ZN-c3 compound nor cause appreciable damage or injury to an animal to which delivery of the composition is intended.

As used herein, a “carrier” refers to a compound that facilitates the incorporation of a compound into cells or tissues. For example, without limitation, dimethyl sulfoxide (DMSO) is a commonly utilized carrier that facilitates the uptake of many organic compounds into cells or tissues of a subject.

As used herein, a “diluent” refers to an ingredient in a pharmaceutical composition that lacks appreciable pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the pH and isotonicity of human blood.

As used herein, an “excipient” refers to an essentially inert substance that is added to a pharmaceutical composition to provide, without limitation, bulk, consistency, stability, binding ability, lubrication, disintegrating ability etc., to the composition. For example, stabilizers such as anti-oxidants and metal-chelating agents are excipients. In an embodiment, the pharmaceutical composition comprises an anti-oxidant and/or a metal-chelating agent. A “diluent” is a type of excipient.

The pharmaceutical compositions described herein can be administered to a human patient per se, or in pharmaceutical compositions where they are mixed with other active ingredients, as in combination therapy, or carriers, diluents, excipients or combinations thereof. Proper formulation is dependent upon the route of administration chosen. Techniques for formulation and administration of the compounds described herein are known to those skilled in the art.

The pharmaceutical compositions disclosed herein may be manufactured in a manner that is itself known, e.g., by means of conventional mixing, dissolving, granulating, dragee-making, levigating, emulsifying, encapsulating, entrapping or tableting processes. Additionally, the ZN-c3 active ingredients is typically contained in an amount effective to achieve its intended purpose, and may be provided as a salt with pharmaceutically compatible counterions.

Multiple art-recognized techniques of administering a ZN-c3 compound, salt and/or composition may be used, but not limited to, oral, rectal, pulmonary, topical, aerosol, injection, infusion and parenteral delivery, including intramuscular, subcutaneous, intravenous, intramedullary injections, intrathecal, direct intraventricular, intraperitoneal, intranasal and intraocular injections. In some embodiments, ZN-c3, or a pharmaceutically acceptable salt thereof, can be administered orally.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions that include ZN-c3 and/or salt, formulated in a compatible pharmaceutical carrier as described herein, may also be prepared, placed in an appropriate container, and labeled for treatment of an indicated condition.

### Uses and Methods of Treatment or Inhibition

Some embodiments described herein relate to a method of treating or inhibiting a cancer in a subject e.g., non-small cell lung cancer, breast cancer, colorectal cancer, ovarian cancer, endometrial cancer, or uterine serous carcinoma (USC), the method comprising administering a therapeutically effective amount of ZN-c3, or a pharmaceutically acceptable salt thereof, to the subject, preferably in accordance with a dosing regimen.

Some embodiments described herein relate to the use of a therapeutically effective amount of ZN-c3, or a pharmaceutically acceptable salt thereof, in the manufacture of a medicament for treating a subject having a cancer e.g., non-small cell lung cancer, breast cancer, colorectal cancer, ovarian cancer, endometrial cancer, or uterine serous carcinoma (USC), preferably in accordance with a dosing regimen. Other embodiments relate to ZN-c3 for use in treating, inhibiting, or ameliorating a cancer in a subject e.g., non-small cell lung cancer, breast cancer, colorectal cancer, ovarian cancer, endometrial cancer, or uterine serous carcinoma (USC), comprising administering a therapeutically effective amount of ZN-c3, or a pharmaceutically acceptable salt thereof, to the subject, preferably in accordance with a dosing regimen. In various embodiments, the dosing regimen comprises an oral administration of the ZN-c3 to the subject. In many of these embodiments, said subject is identified or selected as one having a sensitivity to ZN-c3 and such sensitivity can be determined by analysis of a biological sample from said subject for a modulation of PPP2R1A protein or a gene encoding PPP2R1A such as an over expression of PPP2R1A protein or a transcript encoding a PPP2R1A protein or under expression of PPP2R1A protein or a transcript encoding a PPP2R1A protein. In some embodiments, the modulation of PPP2R1A protein or a gene or transcript encoding a PPP2R1A protein concerns a polymorphism in said PPP2R1A protein or gene or transcript encoding said PPP2R1A protein e.g., any one or more of P179R, S256F or R183W or a gene or transcript encoding a P179R, S256F or R183W polymorphism.

In various embodiments, the therapeutically effective amount of the ZN-c3 administered to the subject is >300 mg per day. For example, in some embodiments, the therapeutically effective amount of the ZN-c3 administered to the subject is about 300 mg per day. In other embodiments, the therapeutically effective amount of the ZN-c3 administered to the subject is about 350 mg per day. In other embodiments, the therapeutically effective amount of the ZN-c3 administered to the subject is about 200 mg per day. In some embodiments, the therapeutically effective amount of the ZN-c3 administered to the subject is >300 mg once daily (QD). In an embodiment, the therapeutically effective amount of the ZN-c3 administered to the subject is about 300 mg once daily (QD). In another embodiment, the therapeutically effective amount of the ZN-c3 administered to the subject is about 350 mg once daily (QD). In still another embodiment, the therapeutically effective amount of the ZN-c3 administered to the subject is about 200 mg once daily (QD). In some embodiments, the therapeutically effective amount of the ZN-c3 administered to the subject is >150 mg two times a day (BID). In an embodiment, the therapeutically effective amount of the ZN-c3 administered to the subject is about 150 mg two times a day (BID). In another embodiment, the therapeutically effective amount of the ZN-c3 administered to the subject is about 175 mg two times a day (BID). In still another embodiment, the therapeutically effective amount of the ZN-c3 administered to the subject is about 100 mg two times a day (BID).

In various embodiments, the dosing regimen comprises a periodic dosing phase during which the amount of the dose and the frequency of ZN-c3 administration to the subject is fixed for a period of time, e.g., 3 or more consecutive days. For example, in an embodiment, the periodic dosing phase comprises a daily dose in the range of about 200 mg per day to about 350 mg per day that is fixed for a period of at least three consecutive days. In an embodiment, the daily dose is administered once daily (QD). In another embodiment, the daily dose is administered in divided doses provided two, three or four times daily. In an embodiment, the periodic dosing phase comprises once daily dosing for a period of at least three consecutive days.

In various embodiments, the dosing regimen comprises an intermittent dosing phase during which the amount of the dose and the frequency of ZN-c3 administration to the subject is changed. In an embodiment, the intermittent dosing phase comprises at least one change in amount of the ZN-c3 administered to the subject on a daily basis. In an embodiment, the intermittent dosing phase comprises at least one rest day, e.g., 1, 2, 3, 4, 5, 6 or 7 rest days. For example, in an embodiment, the dosing regimen comprises a periodic phase during which the ZN-c3 is administered to the subject orally on a once daily basis for five days, following by an intermittent or rest phase during which the ZN-c3 is not administered for two days. Such a dosing regimen, which may be referred to as 5 days on/2 days off, may be repeated or cycled as often as needed, depending on the particular case.

In various embodiments, the subject is a human. In some embodiments, the subject has a cancer e.g., non-small cell lung cancer, breast cancer, colorectal cancer, ovarian cancer, endometrial cancer, or uterine serous carcinoma (USC). In some embodiments, the USC is advanced USC. In some embodiments, the subject has USC that is recurrent USC.

In various embodiments, the subject has been previously treated for a cancer e.g., non-small cell lung cancer, breast cancer, colorectal cancer, ovarian cancer, endometrial cancer, or uterine serous carcinoma (USC) by a prior therapeutic regimen. Various therapeutic regimens are known to those skilled in the art or under active development. In an embodiment, the prior therapeutic regimen can include administering a drug, antibody, CAR T cell, surgery and/or radiation prior to administration of the ZN-c3. For example, in various embodiments, the prior therapy can include at least one selected from Carboplatin, Paclitaxel, Bevacizumab, Trastuzumab, Pembrolizumab, Lenvatinib or Doxorubicin.

The amount of the compound of ZN-c3, or pharmaceutically acceptable salt thereof, that is effective in treating a particular case of cancer in a subject e.g., non-small cell lung cancer, breast cancer, colorectal cancer, ovarian cancer, endometrial cancer, or uterine serous carcinoma (USC) may vary not only with the particulars of the compound or salt selected but also with the route of administration, the nature and/or symptoms of the disease or condition being treated and the age and condition of the patient and will be ultimately at the discretion of the attendant physician or clinician. In cases of administration of a pharmaceutically acceptable salt, dosages may be calculated as the free base. As will be understood by those of skill in the art, in certain situations it may be necessary to administer the ZN-c3 compound, salt or composition in amounts that exceed, or even far exceed, the dosage ranges described herein in order to effectively and aggressively treat particularly aggressive cases of USC.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, the mammalian species treated, the particular ZN-c3 compound, salt or composition employed and the specific use for which these therapies are employed. The determination of effective dosage levels, that is the dosage levels necessary to achieve the desired result, can be accomplished by one skilled in the art using routine methods, for example, human clinical trials, in vivo studies and in vitro studies. For example, useful dosages of ZN-c3, or pharmaceutically acceptable salts thereof, can be determined by comparing their in vitro activity, and in vivo activity in animal models. Such comparison can be done by comparison against an established drug, such as carboplatin and/or paclitaxel.

Dosage amount and interval may be adjusted individually to provide plasma levels of the active moiety which are sufficient to maintain the modulating effects, or minimal effective concentration (MEC). The MEC will vary for each compound but can be estimated from in vivo and/or in vitro data. Dosages necessary to achieve the MEC will depend on individual characteristics and route of administration. However, HPLC assays or bioassays can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the disease or condition to be treated and to the route of administration. The severity of the disease or condition may, for example, be evaluated, in part, by standard prognostic evaluation methods. Further, the dose and perhaps dose frequency, will also vary according to the age, body weight and response of the individual patient. A program comparable to that discussed above may be used in veterinary medicine.

ZN-c3 compounds, salts and compositions disclosed herein can be evaluated for efficacy and toxicity using known methods. For example, the toxicology of ZN-c3 may be established by determining in vitro toxicity towards a cell line, such as a mammalian, and preferably human, cell line. The results of such studies are often predictive of toxicity in animals, such as mammals, or more specifically, humans. Alternatively, the toxicity of particular compounds in an animal model, such as mice, rats, rabbits, dogs or monkeys, may be determined using known methods. The efficacy of a particular compound may be established using several recognized methods, such as in vitro methods, animal models, or human clinical trials. When selecting a model to determine efficacy, the skilled artisan can be guided by the state of the art to choose an appropriate model, dose, route of administration and/or regime.

In some embodiments, a subject's sensitivity to ZN-c3 is determined by analyzing the presence of one or more PPP2R1A polymorphisms e.g., any one or more of P179R, S256F or R183W or a gene or transcript encoding a P179R, S256F or R183W polymorphism. Such polymorphisms can be identified e.g., by using whole genome sequencing methods including Next Generation Sequencing (NGS). In some embodiments, PPP2R1A polymorphisms are identified using polymerase chain reaction (PCR) based methods. In some embodiments, the PCR based methods include allele specific PCR (ASPCR) with Taqman probes, high resolution melt analysis (HRM), digital PCR, co-amplification at lower denaturation temperature PCR (COLD-PCR). In some embodiments, PPP2R1A polymorphisms are identified using mass spectrometry. In some embodiments, the mass spectrometry used can be Matrix-assisted Laser Desorption/ionization time of flight (MALDI-TOF-MS). In some embodiments, PPP2R1A polymorphisms are identified using a small nucleotide polymorphism (SNP) array. In some embodiments, PPP2R1A polymorphisms are identified using a commercial SNaPshot multiplex kit. In some embodiments, PPP2R1A polymorphisms are identified using denaturing high performance liquid chromatography (DHPLC).

In some embodiments, the primers used during the NGS or PCR reactions read through the loci of interest, or the complement thereof.

In some embodiments, the PPP2R1A mutations are hotspot mutations. In some embodiments, the genomic loci for each codon where the hot spots are located are: S256: hg19: chr19: 52,716,321-52,716,323; R183: hg19: chr19: 52,715,981-52,715,983; and/or P179: hg19: chr19: 52,715,969-52,715,971. In some embodiments, these hotspot mutations are found in uterine carcinoma, ovarian cancer, breast cancer, lung cancer, colorectal cancer, prostate cancer, salivary gland cancer, or other cancers.

In some embodiments, modulations in PPP2R1A such as the aforementioned polymorphisms are detected. In some embodiments, the modulations comprise one or more gain of function mutations in PPP2R1A. In some embodiments, the modulations comprise one or more loss of function mutations in PPP2R1A. In some embodiments, the modulations comprise one or more mutations that result in overexpression of PPP2R1A. In some embodiments, the modulations comprise one or more mutations that result in under expression of PPP2R1A.

### Example 1 Dose Response of Cancer Cell Lines Sensitized with PPP2R1A siRNA to ZN-c3

The effect of PPP2R1A knockdown on influencing cancer cell susceptibility to ZN-c3 was evaluated. Various cancer cell lines were transfected with PPP2R1A siRNA or control scrambled siRNA and the cells were treated with a 10 point dose response of ZN-c3 for 72 hours. Percent cell density was calculated using cell titer glow analysis. The PPP2R1A siRNA targeted the following bases Chr.19: 52189802-52226425. SiRNA transfection was started 48 hours prior to treatment with ZN-c3. The ZN-c3 dose response evaluation included ZN-c3 dosages at 10 uM, 3.33 uM, 1.11 uM, 370.3 nM, 123.4 nM, 41.1 nM, 13.7 nM, 4.5 nM, and 1.5 nM.

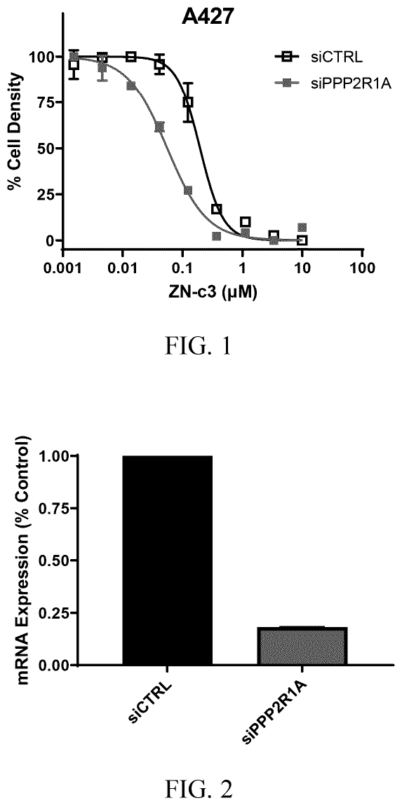

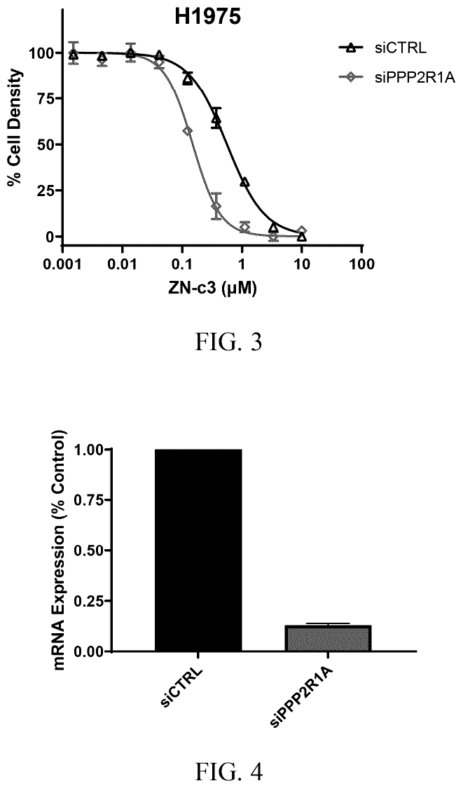

A first non-small cell lung cancer cell line A427 was treated with PPP2R1A siRNA or control siRNA and transfected cells were assessed for sensitivity to ZN-c3 by measurement of reduction in percent cell density (FIG. 1). PPP2R1A siRNA mediated knockdown of PPP2R1A was evaluated by comparison of PPP2R1A expression in conditions treated with siCTRL and siPPPR12A (FIG. 2). Knockdown of PPP2R1A by siRNA was successful, and A427 cells transduced with siPPP2R1A demonstrated a 3.5-fold increase in sensitivity to ZN-c3 (TABLE 1).

TABLE 1

| | siCTRL | siPPP2R1A |
|---|---|---|
| EC50 | 0.1957 | 0.05544 |

A second non-small cell lung cancer cell line H1975 was treated with PPP2R1A siRNA or control siRNA and transfected cells were assessed for sensitivity to ZN-c3 by measurement of reduction in percent cell density (FIG. 3). PPP2R1A siRNA mediated knockdown of PPP2R1A was evaluated by comparison of PPP2R1A expression in conditions treated with siCTRL and siPPPR12A (FIG. 4). Knockdown of PPP2R1A by siRNA was successful, and H1975 cells transduced with siPPP2R1A demonstrated a 3.7-fold increase in sensitivity to ZN-c3 (TABLE 2).

TABLE 2

| | siCTRL | siPPP2R1A |
|---|---|---|
| EC50 | 0.5595 | 0.1500 |

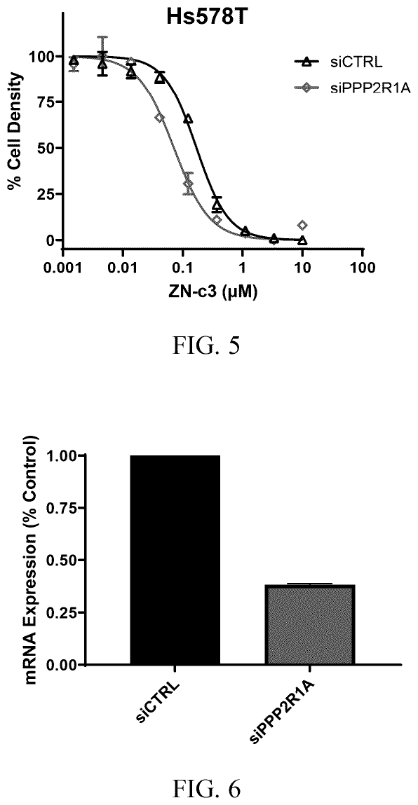

A breast cancer cell line Hs578T was treated with PPP2R1A siRNA or control siRNA and transfected cells were assessed for sensitivity to ZN-c3 by measurement of reduction in percent cell density (FIG. 5). PPP2R1A siRNA mediated knockdown of PPP2R1A was evaluated by comparison of PPP2R1A expression in conditions treated with siCTRL and siPPPR12A (FIG. 6). Knockdown of PPP2R1A by siRNA was successful, and Hs578T cells transduced with siPPP2R1A demonstrated a 2.4-fold increase in sensitivity to ZN-c3 (TABLE 3).

TABLE 3

| | siCTRL | siPPP2R1A |
|---|---|---|
| EC50 | 0.1699 | 0.07133 |

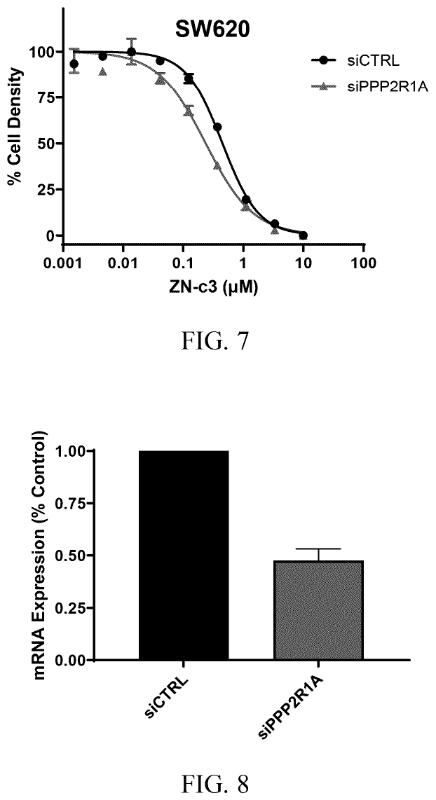

A colorectal cancer cell line SW620 was treated with PPP2R1A siRNA or control siRNA and transfected cells were assessed for sensitivity to ZN-c3 by measurement of reduction in percent cell density (FIG. 7). PPP2R1A siRNA mediated knockdown of PPP2R1A was evaluated by comparison of PPP2R1A expression in conditions treated with siCTRL and siPPPR12A (FIG. 8). Knockdown of PPP2R1A by siRNA was successful, and SW620 cells transduced with siPPP2R1A demonstrated a 1.9-fold increase in sensitivity to ZN-c3 (TABLE 4).

TABLE 4

| | siCTRL | siPPP2R1A |
|---|---|---|
| EC50 | 0.4499 | 0.2364 |

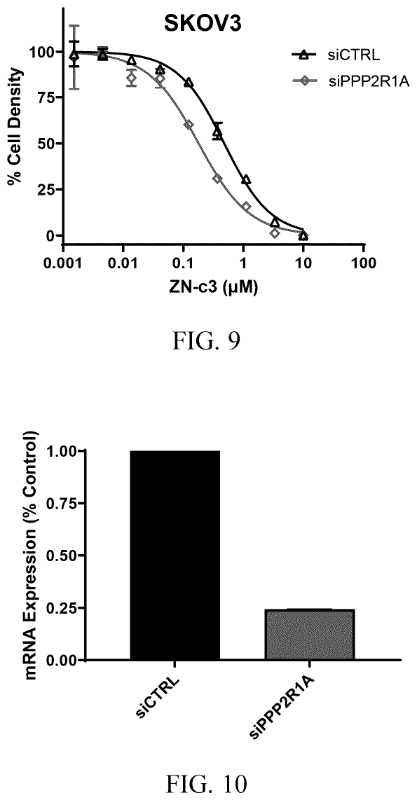

An ovarian cell line SKOV3 was treated with PPP2R1A siRNA or control siRNA and transfected cells were assessed for sensitivity to ZN-c3 by measurement of reduction in percent cell density (FIG. 9). PPP2R1A siRNA mediated knockdown of PPP2R1A was evaluated by comparison of PPP2R1A expression in conditions treated with siCTRL and siPPPR12A (FIG. 10). Knockdown of PPP2R1A by siRNA was successful, and SKOV3 cells transduced with siPPP2R1A demonstrated a 2.7-fold increase in sensitivity to ZN-c3 (TABLE 5).

TABLE 5

| | siCTRL | siPPP2R1A |
|---|---|---|
| EC50 | 0.4804 | 0.1797 |

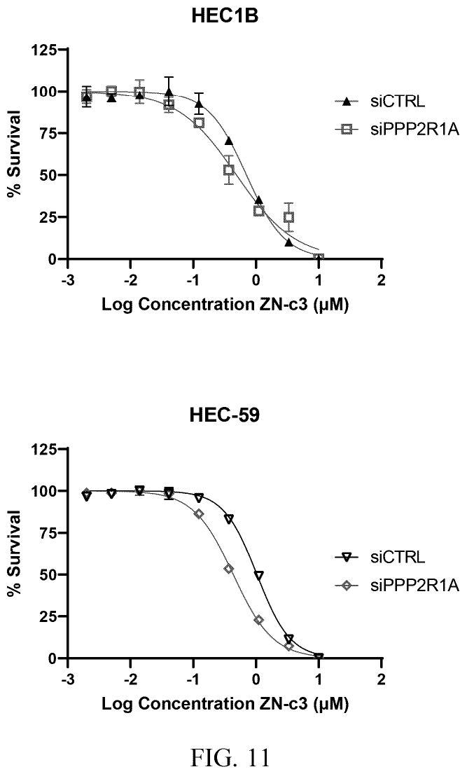

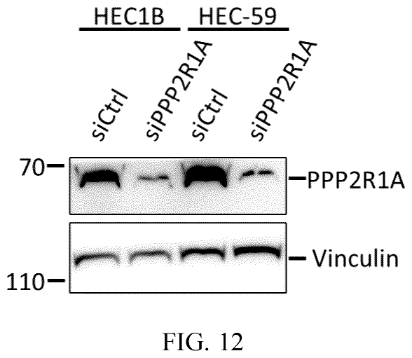

Endometrial cancer cell lines HEC1B and HEC-59 were treated with PPP2R1A siRNA or control siRNA and transfected cells were assessed for sensitivity to ZN-c3 by measurement of reduction in percent cell density (FIG. 11). PPP2R1A siRNA mediated knockdown of PPP2R1A was evaluated by comparison of PPP2R1A protein expression via western blot in conditions treated with siCTRL and siPPPR12A with vinculin used as a control (FIG. 12). Knockdown of PPP2R1A by siRNA was limited in HEC1B cells, due to the missense mutations W257L and L429F within PPP2R1A in this cell line. However, HEC-59 cells possess wild type PPP2R1A and knockdown of PPP2R1A by siRNA was successful in this line. HEC1B cells transfected with PPP2R1A siRNA demonstrated a 1.4-fold increase in sensitivity to ZN-c3 (TABLE 6). HEC-59 cells transfected with PPP2R1A demonstrated a 2.5-fold increase in sensitivity to ZN-c3 (TABLE 6). Thus, these results indicate how knockdown of PPP2R1A plays a critical role in sensitizing cancer cells to ZN-c3 mediated growth inhibition.

TABLE 6

| EC50 | siCTRL | siPPP2R1A |
|---|---|---|
| HEC1B | 0.7072 | 0.4900 |
| HEC-59 | 1.038 | 0.4357 |

### Example 2 Dose Response of Cancer Cell Lines Sensitized with CRISPR Mediated Knockout of PPP2R1A to ZN-c3

Having observed that siRNA mediated knockdown of PPP2R1A sensitized cancer cell lines to treatment with ZN-c3, it was also of interest to evaluate if CRISPR mediated PPP2R1A knockout produced a similar effect. Various cancer cell lines were transfected with a CRISPR guide RNA targeting PPP2R1A or nontargeting guide RNA and the cells were treated with a 10-point dose response of ZN-c3 for 72 hours. The 10-point dose response to ZN-c3 included the same dosages used in Example 1. As in Example 1, no media change was performed during the 72-hour ZN-c3 dose response evaluation period. Fewer than 20 cell passages were completed between the CRISPR mediated knockout and ZN-c3 treatment. CRISPR was performed by targeting PPP2R1A with a PPP2R1A sgRNA sequence. Percent cell density was calculated using cell titer glow analysis.

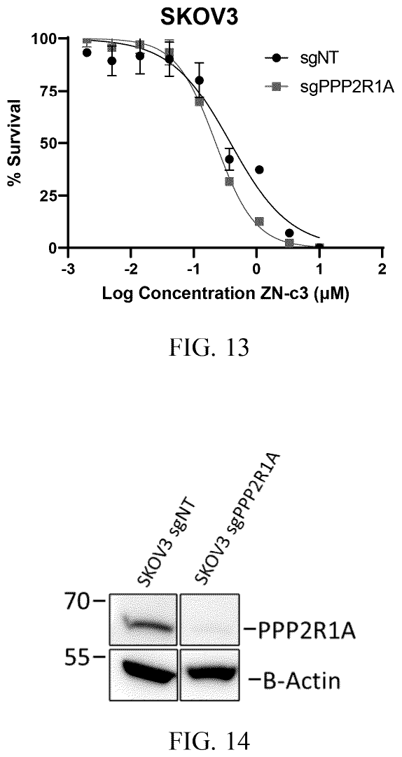

The ovarian cancer cell line SKOV3 was treated with a CRISPR guide RNA targeting PPP2R1A or nontargeting guide RNA and transfected cells were assessed for sensitivity to ZN-c3 by measurement of reduction in percent cell density (FIG. 13). CRISPR mediated knockout of PPP2R1A was evaluated by comparison of PPP2R1A protein expression via Western blot in conditions treated with nontargeting guide RNA and PPPR12A targeting CRISPR guide RNA with beta actin as a control (FIG. 14). Knockout of PPP2R1A by CRISPR was successful, and SKOV3 cells transduced with PPP2R1A targeting guide RNA demonstrated a 1.7-fold increase in sensitivity to ZN-c3 (TABLE 7).

TABLE 7

| | sgNT | sgPPP2R1A |
|---|---|---|
| EC50 | 0.3856 | 0.2239 |

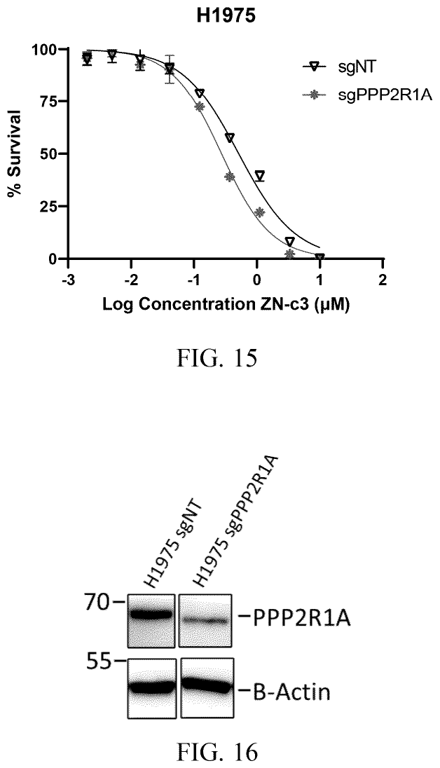

The lung cancer cell line H1975 was treated with a CRISPR guide RNA targeting PPP2R1A or nontargeting guide RNA and transfected cells were assessed for sensitivity to ZN-c3 by measurement of reduction in percent cell density (FIG. 15). CRISPR mediated knockout of PPP2R1A was evaluated by comparison of PPP2R1A expression in conditions treated with nontargeting guide RNA and PPPR12A targeting CRISPR guide RNA (FIG. 16). Knockout of PPP2R1A by CRISPR was successful, and H1975 cells transduced with PPP2R1A targeting guide RNA demonstrated a 1.9-fold increase in sensitivity to ZN-c3 (TABLE 8).

TABLE 8

| | sgNT | sgPPP2R1A |
|---|---|---|
| EC50 | 0.5155 | 0.2752 |

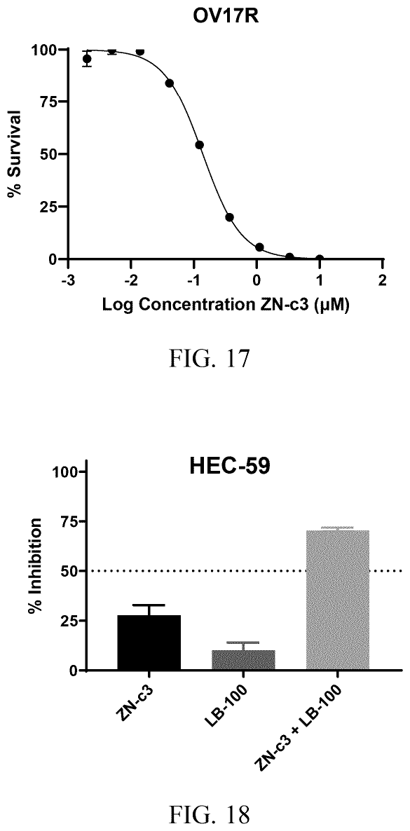

The ovarian cancer cell line OV17R was assessed for sensitivity to ZN-c3 by measurement of reduction in percent cell density (FIG. 17). OV17R cells possess a S256F hotspot missense mutation in PPP2R1A, thus rendering these cells highly susceptible to ZN-c3 without the need for an additional mechanism for PPP2R1A knockdown or knockout (TABLE 9).

TABLE 9

| | OV17R |
|---|---|
| EC50 | 0.1391 |

### Example 3 Response of Cancer Cell Lines to Treatment with ZN-c3, LB-100, or ZN-c3+LB-100

Following observations that both knockdown and knockout of PPP2R1A could sensitize cancer cells to Zn-C3, the effects of pharmacological inhibition of PPP2R1A with the catalytic inhibitor LB-100 were evaluated. Various cancer cell lines were treated with ZN-c3 alone, the PP2A catalytic inhibitor LB-100 alone, or the combination of ZN-c3+LB-100. As in Example 1 and Example 2, the treatment period was 72 hours and percent inhibition was calculated using cell titer glow analysis.

The endometrial cancer cell line HEC-59 was assessed for sensitivity to ZN-c3 alone, LB-100 alone, or the combination of ZN-c3+LB-100 (FIG. 18). Doses of ZN-c3 and LB-100 are shown in TABLE 10. ZN-c3 treatment alone was capable of achieving approximately 25% inhibition, while LB-100 treatment achieved approximately half of that value. However, combination therapy with both ZN-c3 and LB-100 was noticeably more effective and reached almost 75% inhibition.

TABLE 10

| Compound | Dose | |
|---|---|---|
| ZN-c3 | 400 | nM |
| LB-100 | 1 | μM |

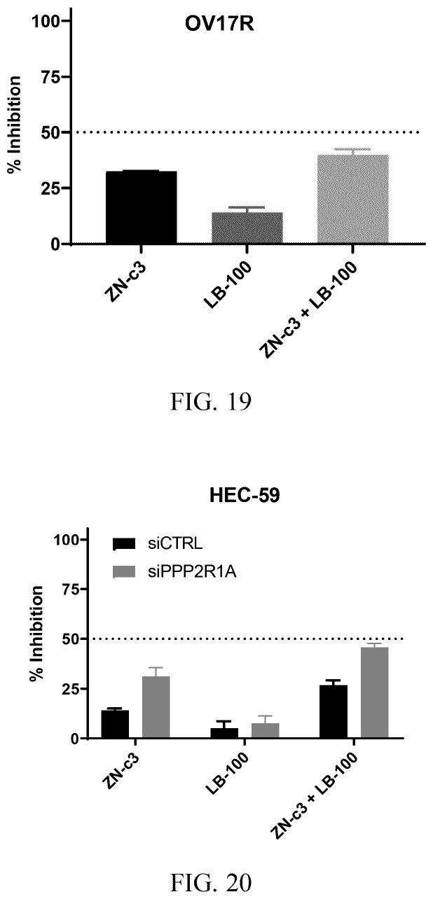

The ovarian cancer cell line OV17R was assessed for sensitivity to ZN-c3 alone, LB-100 alone, or the combination of ZN-c3+LB-100 (FIG. 19). Doses of ZN-c3 and LB-100 are shown in TABLE 11. ZN-c3 treatment alone was capable of achieving approximately 30% inhibition, while LB-100 treatment achieved approximately 12% inhibition. Combination therapy with both ZN-c3 and LB-100 demonstrated a modest increase over ZN-C3 alone to approximately 40% inhibition. The small increase in percent inhibition seen in combination therapy was likely a result of the PPP2R1A missense mutation S256F present in OV17R that is not present in the other cancer lines tested which possess wild type PPP2R1A.

TABLE 11

| Compound | Dose | |
|---|---|---|
| ZN-c3 | 75 | nM |
| LB-100 | 1 | μM |

The endometrial cancer cell line HEC-59 was assessed for sensitivity to ZN-c3 alone, LB-100 alone, or the combination of ZN-c3+LB-100 following treatment with siCTRL or siPPP2R1A (FIG. 20). Doses of ZN-c3 and LB-100 are shown in TABLE 12. ZN-c3 treatment alone was capable of achieving approximately 12% inhibition, while LB-100 treatment achieved approximately half of that value in cells treated with siCTRL. However, combination therapy with both ZN-c3 and LB-100 was noticeably more effective and exceeded 25% inhibition in siCTRL treated cells. Significantly, the percent inhibition achieved by ZN-c3 treatment alone and by ZN-c3 in combination with LB-100 was approximately doubled in cells that had been treated with siPPP2R1A compared to cells treated with siCTRL. The percent inhibition by LB-100 alone was only slightly enhanced in cells with PPP2R1A knockdown. These data evidence that pairing pharmacological inhibition (LB-100) with Zn-C3 treatment on a background with reduced PPP2R1A expression provides a strong combinatorial approach to cancer treatment.

TABLE 12

| Compound | Dose | |
|---|---|---|
| ZN-c3 | 300 | nM |
| LB-100 | 1 | μM |

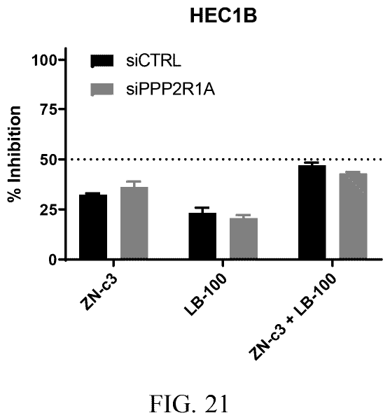

The endometrial cancer cell line HEC1B was also assessed for sensitivity to ZN-c3 alone, LB-100 alone, or the combination of ZN-c3+LB-100 following treatment with siCTRL or siPPP2R1A (FIG. 21). Doses of ZN-c3 and LB-100 are shown in TABLE 13. ZN-c3 treatment alone was capable of achieving approximately 30% inhibition, while LB-100 treatment achieved 25% inhibition in cells treated with siCTRL. Combination therapy with both ZN-c3 and LB-100 was more effective than either treatment individually, and approached 50% inhibition in siCTRL treated cells. The percent inhibition achieved by ZN-c3 treatment alone and by ZN-c3 in combination with LB-100 was approximately the same regardless of whether cells had been treated with siPPP2R1A or with siCTRL. This result is similar to evaluations of HEC1B in the previous examples, where the missense mutations W257L and L429F within PPP2R1A in this cell line limited induction of sensitivity to ZN-c3.

TABLE 13

| Compound | Dose | |
|---|---|---|
| ZN-c3 | 200 | nM |
| LB-100 | 1 | μM |

### Example 4 Clinical Trial of ZN-c3 in Patients with PPP2R1A Genetic Variants

Having observed that genetic ablation, and pharmacological inhibition of PPP2R1A sensitizes cancer cell lines to ZN-c3, the next objective was to investigate the clinical response to ZN-c3 in human patients with hotspot mutations in PPP2R1A. Five USC patients in which prior genomic profiling information for PPP2R1A was known were evaluated as part of a clinical study. Three patients P179R variant of PPP2R1A, and two patients possessed the S256F variant (TABLE 14). All patients were evaluable for clinical response by at least one tumor assessment by imaging using computed tomography (CT) and/or magnetic resonance imaging (MRI). Four out of five patients (80%) showed clinical benefit from ZN-c3. The objective response rate of patients with PPP2R1A mutations was 20%.

For subject number 1, a starting dose of 300 mg QD ZN-c3 was administered, dosage was reduced to 5 days on and 2 days off on day 44, dosage was further reduced to 200 mg QD on day 51, and the last dose was provided on day 230.

For subject number 2, a starting dose of 300 mg QD ZN-c3 was administered, dosage was reduced to 200 mg QD on day 36, the dosage was further reduced to 200 mg 5 days on and 2 days off and on day 50, and the last dose was provided on day 84.

For subject number 3, a starting dose of 300 mg QD ZN-c3 was administered, dosage was reduced to 200 mg QD on day 71, and the last dose was provided on day 172.

For subject number 4, a starting dose of 300 mg QD ZN-c3 was administered, dosage was not reduced during the study, and the last dose was provided on day 64.

For subject number 5, a starting dose of 300 mg QD ZN-c3 was administered, dosage was reduced to 150 mg BID on day 36, and the last dose was provided on day 62.

TABLE 14

| Subject nr. | PPP2R1A variant | Best overall response | |
|---|---|---|---|
| 1 | S256F | cPR | (−43%) |
| 2 | P179R | SD | (−26%) |
| 3 | P179R | SD | (−12%) |
| 4 | P179R | SD | (−7%) |

| | | |
|---|---|---|
| 5 | S256F | PD |

cPR = Confirmed partial response. SD = Stable disease. PD = Progressive disease.

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure.

What is claimed is:

1. A method of identifying or selecting a subject that has a sensitivity to the drug ZN-c3, comprising:
identifying a modulation in the protein phosphatase 2 scaffold subunit Alpha (PPP2R1A) protein or a gene encoding said PPP2R1A protein, such as one or more polymorphisms, which confers a sensitivity to the drug ZN-c3, preferably a polymorphism selected from P179R, S256F or R183W or a nucleic acid encoding a polymorphism selected from P179R, S256F or R183W, in a biological sample obtained from said subject or individual; and
selecting or identifying said subject as one having a sensitivity to the drug ZN-c3, when said modulation, such as said any one or more polymorphisms in the PPP2R1A gene or protein, is identified.

2. The method of claim 1, further comprising administering ZN-c3 to said subject when said modulation or polymorphism is identified in said biological sample.

3. The method of any one of claims 1 or 2, wherein said subject has a cancer.

4. The method of claim 3, wherein the cancer comprises non-small cell lung cancer, breast cancer, colorectal cancer, ovarian cancer, uterine serous carcinoma (USC), or endometrial cancer.

5. The method of any one of claims 2-4, wherein regression or inhibition of said cancer is greater than 7%, such as 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

6. The method of any one or claims 2-5 further comprising administration of an agent or combination of agents to said subject, which inhibit the PPP2R1A gene or protein.

7. The method of claim 6, wherein said agent or combination of agents comprise the PP2A catalytic inhibitor LB-100.

8. The method of any one of claims 1 through 7, wherein the modulation of PPP2R1A comprises one or more gain of function mutations.

9. The method of any one of claims 1 through 7, wherein the modulation of PPP2R1A comprises one or more loss of function mutations.

10. The method of any one of claims 1 through 7, wherein the modulation of PPP2R1A comprises one or more mutations that result in overexpression of PPP2R1A.

11. The method of any one of claims 1 through 7, wherein the modulation of PPP2R1A comprises one or more mutations that result in under expression of PPP2R1A.

12. A method of inhibiting, ameliorating, or treating a cancer or sequela thereof in a subject, the method comprising:
identifying a modulation in the protein phosphatase 2 scaffold subunit Alpha (PPP2R1A) protein or a gene encoding said PPP2R1A protein, wherein said polymorphism confers a sensitivity to the drug ZN-c3, such as a polymorphism selected from P179R, S256F or R183W or a nucleic acid encoding a polymorphism selected from P179R, S256F or R183W, in a biological sample obtained from said subject; and
administering ZN-c3 to said subject when said polymorphism is identified in said biological sample.

13. The method of claim 12, wherein the cancer comprises non-small cell lung cancer, breast cancer, colorectal cancer, ovarian cancer, uterine serous carcinoma (USC), or endometrial cancer.

14. The method of claim 12 or 13, wherein regression or inhibition of said cancer is greater than 7%, such as 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

15. The method of any one or claims 12-14, wherein an agent or combination of agents, which inhibit the PPP2R1A gene or protein are additionally provided to said subject.

16. The method of claim 15, wherein said agent or combination of agents comprise the PP2A catalytic inhibitor LB-100.

17. The method of any one of claims 12 through 16, wherein the modulation of PPP2R1A comprises one or more gain of function mutations.

18. The method of any one of claims 12 through 16, wherein the modulation of PPP2R1A comprises one or more loss of function mutations.

19. The method of any one of claims 12 through 16, wherein the modulation of PPP2R1A comprises one or more mutations that result in overexpression of PPP2R1A.

20. The method of any one of claims 12 through 16, wherein the modulation of PPP2R1A comprises one or more mutations that result in under expression of PPP2R1A.

21. The compound ZN-c3 for use in inhibiting, ameliorating, or treating a cancer or sequela thereof in a subject identified as having a modulation in the protein phosphatase 2 scaffold subunit Alpha (PPP2R1A) protein or a gene encoding said PPP2R1A protein, such as a polymorphism, which confers a sensitivity to the drug ZN-c3, e.g., a polymorphism selected from P179R, S256F or R183W or a nucleic acid encoding a polymorphism selected from P179R, S256F or R183W, in a biological sample obtained from said subject.

22. The compound ZN-c3 for use according to claim 21, wherein the cancer comprises non-small cell lung cancer, breast cancer, colorectal cancer, ovarian cancer, uterine serous carcinoma (USC), or endometrial cancer.

23. The compound ZN-c3 for use according to claim 21, wherein regression or inhibition of said cancer is greater than 7%, such as 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

24. The compound ZN-c3 for use according to any one of claims 21-23, wherein an agent or combination of agents, which inhibit the PPP2R1A gene or protein are additionally provided to said subject.

25. The compound ZN-c3 for use according to claim 24, wherein said agent or combination of agents comprise the PP2A catalytic inhibitor LB-100.

26. The compound ZN-c3 for use according to any one of claims 21 through 25, wherein the modulation of PPP2R1A comprises one or more gain of function mutations.

27. The compound ZN-c3 for use according to any one of claims 21 through 25, wherein the modulation of PPP2R1A comprises one or more loss of function mutations.

28. The compound ZN-c3 for use according to any one of claims 21 through 25, wherein the modulation of PPP2R1A comprises one or more mutations that result in overexpression of PPP2R1A.

29. The compound ZN-c3 for use according to any one of claims 21 through 25, wherein the modulation of PPP2R1A comprises one or more mutations that result in under expression of PPP2R1A.

30. A method of inhibiting, ameliorating, or treating a cancer or sequela thereof in a subject, the method comprising:
administering an agent or combination of agents, which inhibit the PPP2R1A gene or protein, to said subject; and
administering ZN-c3 to said subject.

31. The method of claim 30, wherein said agent or combination of agents, which inhibit the PPP2R1A gene or protein, are provided to said subject separately.

32. The method of claim 30 or 31, wherein said agent or combination of agents, which inhibit the PPP2R1A gene or protein, are provided to said subject before administration of ZN-C3.

33. The method of claim 30, wherein said agent or combination of agents, which inhibit the PPP2R1A gene or protein, are provided to said subject within 1, 2, 3, 4, 5, 6, 7, 8, 9, 10, 11, 12, 13, 14, 15, 16, 17, 18, 19, or 20 minutes apart.

34. The method of any one of claims 30-33, wherein the cancer comprises non-small cell lung cancer, breast cancer, colorectal cancer, ovarian cancer, uterine serous carcinoma (USC), or endometrial cancer.

35. The method of any one of claims 30-34, wherein regression or inhibition of said cancer is greater than 7%, such as 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

36. The method of any one of claims 30-35, wherein said agent or combination of agents comprise the PP2A catalytic inhibitor LB-100.

37. A product combination comprising the compound ZN-c3 and an agent or combination of agents, which inhibit the PPP2R1A gene or protein for use in inhibiting, ameliorating, or treating a cancer or sequela thereof in a subject.

38. The product combination for use according to claim 37, wherein the cancer comprises non-small cell lung cancer, breast cancer, colorectal cancer, ovarian cancer, uterine serous carcinoma (USC), or endometrial cancer.

39. The product combination for use according to any one of claims 37 or 38, wherein regression or inhibition of said cancer is greater than 7%, such as 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, or more.

40. The product combination for use according to any one of claims 37-39, wherein said agent or combination of agents comprise the PP2A catalytic inhibitor LB-100.

41. The method of any one of claims 1-20 or the compound ZN-c3 for use according to any one of claims 21-29, wherein the presence of said polymorphism is determined using Next Generation Sequencing (NGS), sequencing, Polymerase Chain Reaction (PCR), Loop-mediated isothermal amplification, Recombinase polymerase amplification, or antibody detection.

42. A method of treating cancer, comprising administering to a subject in need thereof, a therapeutically effective amount of ZN-c3 and LB-100.

43. The method of claim 42, wherein the cancer is endometrial cancer, ovarian cancer, uterine serous carcinoma, non-small cell lung cancer, breast cancer or colorectal cancer.

44. The method of claim 43, wherein the cancer is endometrial cancer.

45. The method of claim 43, wherein the cancer is ovarian cancer.

46. The method of claim 43, wherein the cancer is uterine serous carcinoma.

47. The method of claim 42, wherein LB-100 is administered to the subject separately from ZN-c3.

48. The method of claim 42, wherein the therapeutically effective amount of ZN-c3 is about 300 mg or greater.

49. The method of claim 42, wherein the therapeutically effective amount of ZN-c3 is about 200 mg.

50. The method of claim 42, wherein the ZN-c3 is administered in two, three or four divided doses per day.

51. The method of claim 42, wherein ZN-c3 is administered in an intermittent dosing regimen.

52. The method of claim 42, wherein the subject has a polymorphism in a gene encoding phosphatase 2 scaffold subunit Alpha (PPP2R1A) or encoded protein.

53. The method of claim 52, wherein the polymorphism is selected from P179R, S256F, and R183W.

54. A combination comprising ZN-c3 and LB-100 for use in treating a cancer.

55. Use of a combination of ZN-c3 and LB-100 in the manufacture of a medicament for treating a cancer.

56. Use of ZN-c3 in the manufacture of a medicament for treating a cancer in combination with LB-100.

57. Use of LB-100 in the manufacture of a medicament for treating a cancer in combination with ZN-c3.

58. The combination of claim 54 or the use of any one of claims 55-57, wherein the cancer is endometrial cancer, ovarian cancer, uterine serous carcinoma, non-small cell lung cancer, breast cancer or colorectal cancer.
